(19) United States
(12) Patent Application Publication
(10) Pub. No.: US 2007/0239166 A1
(43) Pub. Date: Oct. 11, 2007

(54) Surgical Device for Anterolateral and Posterolateral Reconstruction

(76) Inventors: David A. McGuire, Anchorage, AK (US); Jeffrey C. Wolchok, Salt Lake City, UT (US); Steven D. Hendricks, Anchorage, AK (US)

Correspondence Address: BROMBERG & SUNSTEIN LLP, 125 SUMMER STREET, BOSTON, MA, 02110-1618, US

(21) Appl. No.: 11/740,607

(22) Filed: Apr. 26, 2007

Related U.S. Application Data
(63) Continuation-in-part of application No. 11/125,808, filed on May 10, 2005.
(60) Provisional application No. 60/569,987, filed on May 11, 2004.

(51) Int. Cl. A61F 5/01

(52) U.S. Cl. 606/86

(57) ABSTRACT

An isometer for determining isometric graft tunnel placement for lateral side surgical augmentation or reconstruction for anterolateral or posterior lateral rotary knee instability. This graft tunnel placement isometry assessment and measurement device is designed to be used with a graft and an extra-articular technique that restores rotatory, lateral, and anterior/posterior knee stability when used by itself or in combination with intra-articular ACL/PCL reconstruction. The isometer includes an elongated body with a slider rod that slides horizontally within the body and a cannulated fixed stylus attached transversely to the first end of the elongated body. Within that cannulated fixed stylus is a guide pin that slides freely but that can also be fixed using a fixed-stylus set screw. Similarly, a cannulated movable stylus is attached transversely to the first end of the slider rod, and within that cannulated movable stylus is a guide pin that slides freely but that can also be fixed using a movable-stylus set screw. There is a pointer encircling the slider rod that slides with the slider rod, but whose position along the slider rod can also be adjusted manually. The slider rod may also have a rotatable member that allows each end of the slider rod to rotate relative to the other end. The rotatable member obviates slider rod impingement during operation. To determine rotary knee isometry during knee reconstructive surgery, the guide pins are positioned over previously identified lateral reconstruction tunnel sites. The guide pins are then driven into the bone tunnel sites and the knee put through a full range of motion by flexing and extending the knee while the isometer position is maintained and the pointer movement is monitored. If movement of the pointer is unacceptably large, the guide pins are re-set, re-positioned and re-driven into different pre-identified lateral reconstruction tunnel sites, and pointer movement during full range motion is again monitored until movement is acceptably small.

### PRIORITY

This application is a continuation in part of utility patent application Ser. No. 11/125,808, entitled “A Surgical Device for Anterolateral Reconstruction” filed May 10, 2005, attorney docket 1464/156, which claims priority from U.S. provisional patent application, Ser. No.: 60/569,987, filed on May 11, 2004 entitled “Surgical Device for Anterolateral Reconstruction”, both of which are incorporated by reference herein in their entirety.

### BACKGROUND OF THE INVENTION

The present invention relates to surgical devices and procedures used in the field of knee surgery, particularly anterolateral and posterolateral reconstruction in combination with reconstruction of the anterior cruciate ligament (ACL) and posterior cruciate ligament (PCL) of the knee.

Three situations have been identified that frequently lead to severe anterolateral and/or posterolateral rotary instability in the knee. In such cases, reconstruction should be considered in combination with ACL and/or PCL reconstruction. The three situations are: 1) when patients present with a failed isolated ACL or PCL reconstruction in which the tunnels, the graft, and the rehabilitation all appear to have been properly done; 2) when patients who have had a previous lateral reconstruction in conjunction with an ACL or PCL reconstruction are persistently unstable; and 3) when patients have suffered a knee dislocation. Other situations may exist, and should be determined on an individual basis.

Failure to recognize such severe anterolateral and posterolateral rotary instability and to perform the appropriate extra-articular reconstruction is an under-recognized cause of ACL and PCL reconstruction failure. Thus, a means for identifying prospective ACL and PCL reconstruction failure candidates is needed, for example, a mechanism for assessing lateral rotary instability/stability.

The Lachman's flexion rotation drawer tests can be used to assist in the diagnoses of severe lateral rotary instability, helping to identify candidates that should be considered for anterior lateral (or posterior lateral) reconstruction in combination with the ACL (or PCL) reconstruction. Those patients with positive Lachman's flexion rotation drawer tests with grades of at least 2+ have proved to be grossly unstable. Once identified, a procedure and/or device for determining and restoring lateral isometry within an acceptable range during ALR reconstruction is needed. Presently, surgeons rely on “free hand” techniques to determine and restore lateral isometry during ALR reconstructions. These “free hand” methods are time consuming and not always accurate. A faster, more reliable and more reproducible method and apparatus for determining and restoring lateral isometry would thus benefit both surgeons and patients.

### SUMMARY OF THE INVENTION

The present invention as embodied is directed to a surgical device and method of using the surgical device, referred to as an isometer, for restoring rotary and anterior or posterior knee stability when used in combination with an intra-articular ACL or PCL reconstruction or with an extra-articular lateral side augmentation procedure. The isometer eliminates the variables in “free hand” determination and restoration of lateral rotary stability, and also decreases the time needed to determine lateral rotary isometry.

The isometer includes an elongated body with a slider rod that slides horizontally within the body along a longitudinal slot running nearly the length of the rod. A cannulated fixed stylus is attached transversely to the first end of the elongated body and within that cannulated fixed stylus is a fixed-stylus guide pin that slides freely and but that can be fixed using a fixed-stylus set screw. A cannulated movable stylus is attached transversely to the first end of the slider rod, and within that cannulated movable stylus is a movable-stylus guide pin that slides freely within the cannulated movable stylus but that can be fixed using a movable-stylus set screw. There is a pointer encircling the slider rod that slides with the slider rod, but whose position along the slider rod can also be adjusted manually.

In use, the guide pins are directed into the fixed and movable styli, advanced until the tips protrude beyond the fixed and movable stylus ends, and then immobilized within the styli by tightening the set screws. Once immobilized within the fixed and movable styli, the immobilized guide pins are positioned over previously identified lateral reconstruction tunnel sites such that the fixed stylus and guide pin are positioned over a femoral tunnel site and the movable stylus and guide pin are positioned over a tibial tunnel site. Of course, it is understood that the placement site of pin positioning may be reversed, with the immobilized stylus and guide pins being positioned over a femoral tunnel site and the movable stylus and guide pins being positioned over a tibial tunnel site.

The guide pins are next driven into the bone tunnel sites. In another variation, the guide pins may be first driven into the bone tunnel sites, and then the isometer seated on the guide pins. After the pins are inserted into the bone tunnel sites, and the pins are locked into position using a locking mechanism, such as the previously described set screws, the knee is put through a full range of motion by flexing and extending the knee while the isometer position is maintained. During these full range movements, the movement of the pointer in relation to the isometer body is monitored. If movement of the pointer is unacceptably large (e.g., greater than 2 mm), the fixed, movable, or both guide pins are re-set, re-positioned and re-driven into different pre-identified lateral reconstruction tunnel sites and the movement of the pointer during full range motion is again monitored until the pointer movement is acceptably small. The determination of the maximum distance allowable is surgeon dependent and depends on the patient's anatomy. In ideal or near ideal circumstances, the maximum movement would be no greater than 2 mm.

At this time, the set screws for the fixed and movable guide pins within their respective styli are loosened and the isometer is removed leaving the guide pins in place for completion of the reconstructive surgery. Once the isometer, is removed from the guide pins, a cannulated drill can be seated on a guide pin and a femoral tunnel can be drilled. Similarly, the cannulated drill can be seated on the other guide pin and a tibial tunnel can be drilled.

The isometer of the present invention substantially eliminates the guess-work and skill-intensive techniques inherent in “free-hand” isometry determinations during ALR/ACL combination reconstructive knee surgery, and greatly standardizes the process for such determinations.

In other embodiments of the present invention, an isometer for use during lateral augmentation or reconstruction surgery for determining if there is lateral isometry includes a cannulated body, a slider rod, and a first and second stylus. The slider rod has a first end and a second end, and passes through the cannulated body. The slider rod is free to slide within the cannulated body. The first stylus is attached to the cannulated body, and the second stylus is attached to the slider.

The slider rod may also include a rotatable coupling located between the first end and second end. The rotatable coupling allows the first end and second end to rotate relative to each other about an axis. The axis of rotation may lie along the length of the slider rod. In some embodiments, the rotatable coupling allows the first-end and second end to rotate 360 degrees. In other embodiments, the rotatable coupling allows the first end and second end to rotate more or less than 360 degrees. The rotatable coupling may be a variety of elements including, but not limited to a bushing.

The body may also have an opening that exposes the slider rod. The slider rod may also include a pointer which is visible through the opening on the side of the body. In such embodiments, the body may have indicia for measuring the position of the pointer.

The first stylus and second stylus may include points for insertion into a bone, and may also be cannulated. A first guide pin can pass through the first cannulated stylus, and a second guide pin can pass through the second cannulated stylus. The first stylus and second stylus may each also include a restraint for securing the guide pins within the cannulated stylus. The restraints may be configured to release the first and second styli when the guide pins are inserted into a bone such that the isometer can be removed, leaving the guide pins inserted in the bone. The restraints may include a set screw which can be tightened to secure the first and second guide pins and loosened to release the first and second guide pins.

The first stylus can pass through a hole at a first end of the sliding rod, and the second stylus may pass through a slot running part of the length of the slider rod such that the slider rod can slide around the second stylus. The rotatable coupling may be located between the hole at the first end and an end of the slot.

In accordance with still other embodiments, a method of using an isometer can determine a position of isometry during knee surgery. The method first inserts a first guide pin into a moveable stylus such that the tip of the guide pin protrudes past an end of the moveable stylus, and inserts a second guide pin into a stationary stylus such that the tip of the second guide pin protrudes past an end of the stationary stylus. The first and second guide pins are then secured in place by tightening set-screws located in the moveable and stationary styli. The method then positions the isometer such that the guide pins are located over previously identified tunnel sites, and drives the guide pins into the tunnel sites.

Once the isometer is in place, a doctor or other medical personnel may flex and extend the knee such that a slider rod in the isometer moves relative to the isometer body. The slider rod may include a rotatable coupling that permits a first end and a second end of the slider arm to rotate relative to one another, compensating for any deviation from parallel between the first and second guide pin. During flexing and extending, the doctor monitors the movement of a pointer located on a slider rod of the isometer in relation to the isometer body.

The doctor may position the first guide pin over a tunnel site located in the femur and the second guide pin over a tunnel site located in the tibia. Alternatively, the doctor can position the first guide pin over a tunnel site located in the tibia and the second guide pin over a tunnel site located in the tibia. The guide pins may be driven into the tunnel sites to a depth of ⅛ to ¼ of an inch.

In some embodiments, the doctor may re-position the first and second styli if the pointer movement is unacceptably large and repeat the positioning process until the pointer movement is acceptable. If the pointer movement is acceptably small, the doctor may loosen the set-screws in the moveable and stationary styli, and remove the isometer, leaving the guide pins in place. The pointer movement may be acceptably small when the pointer movement is 2 mm or less.

In accordance with another embodiment, a method of using an isometer to determine a position of isometry during knee surgery includes driving a first guide pin into a predetermined tunnel site in a femur, driving a second guide pin into a predetermined tunnel site in a tibia, and placing the isometer over the guide pins. The isometer may be placed such that a moveable stylus located on the isometer slides over one guide pin and a stationary stylus located on the isometer slides over the other guide pin. The tip of the guide pins may protrude past the ends of the styli. The method then secures the first and second guide pins in place by tightening set-screws located in the moveable and stationary styli.

Once the guide pins are secured, a doctor may flex and extend the knee such that a slider rod of the isometer moves relative to an isometer body. The slider rod includes a rotatable coupling that permits a first end and a second end of the slider arm to rotate relative to one another, compensating for any deviation from parallel between the first and second guide pin. The doctor can then monitor the movement of a pointer located on the slider arm in relation to the isometer body.

The doctor may place the moveable stylus over the first guide pin and the stationary stylus over the second guide pin. Alternatively, the doctor may place the stationary stylus over the first guide pin and the moveable stylus over the second guide pin. If movement is unacceptably large, the doctor may re-position the first and second guide pins and repeat the process until the pointer movement is acceptable. If the pointer movement is acceptably small, the doctor can loosen the set-screws in the moveable and stationary styli, and removing the isometer, leaving the guide pins in place. In some embodiments, the pointer movement is acceptably small when the pointer movement is 3 mm or less.

### BRIEF DESCRIPTION OF THE DRAWINGS

FIG. 1 illustrates an isometer device of the present invention;

FIG. 1B illustrates an isometer device of the present invention with a rotatable coupling on the slider rod;

FIG. 2 illustrates an enlarged view of the elongated isometer body;

FIG. 3A illustrates a top view of the slider rod;

FIG. 3B illustrates a side view of the slider rod;

FIG. 3C illustrates a top view of an alternative embodiment of the slider rod in which the rod includes a rotatable coupling;

FIG. 3D illustrates a side view of the alternative embodiment of the slider rod in which the rod includes a rotatable coupling;

FIG. 4A is a flow chart showing the steps for using the isometer;

FIG. 4B is a flow chart showing the steps for using an isometer with rotatable coupling;

FIG. 5 shows a bone model with proposed placement of the tibial and femoral tunnels;

FIG. 6A shows the bone model of FIG. 5 superimposed on a knee during surgery;

FIG. 6B shows the bone model of FIG. 5 with both guide pins inserted into the placement positions;

FIG. 7A shows the placement of the isometer over the guide pins on the lateral side of the knee for determining if there is isometry for the tunnel sites; and

FIG. 7B shows the placement of the isometer with rotatable coupling over the guide pins on the lateral side of the knee for determining if there is isometry for the tunnel sites.

### DETAILED DESCRIPTION OF THE INVENTION

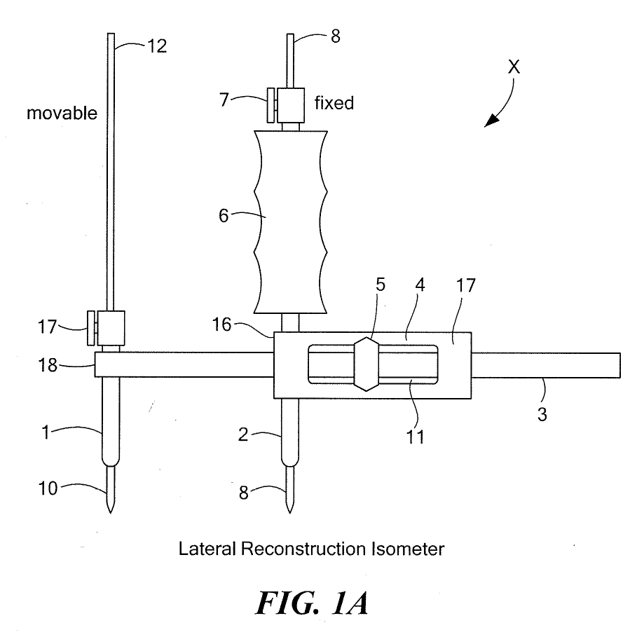

Referring now to FIG. 1, an isometer X having an elongated cannulated body 4, having a first end 16 and a second end 17, that encases a slider rod 3 with a pointer 5, further having a fixed stylus 2 and corresponding guide pin 8, and a movable stylus 1 and corresponding guide pin 10, is designed for use in determining lateral isometry for restoring rotary and anterior and/or posterior knee stability in combination with ACL and/or PCL reconstruction.

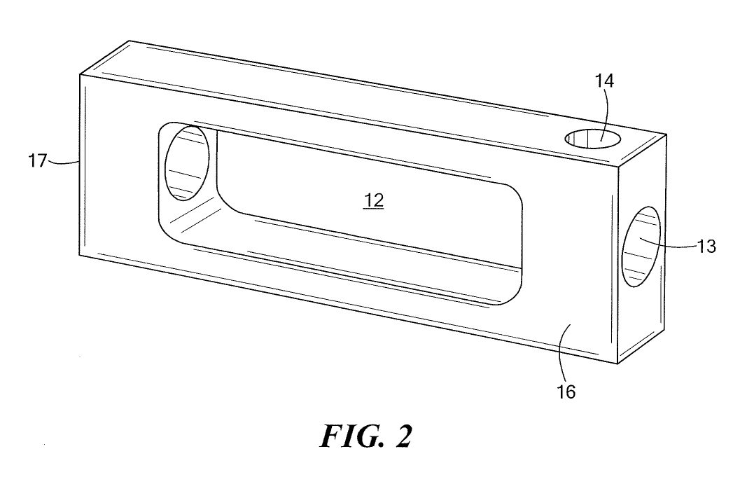

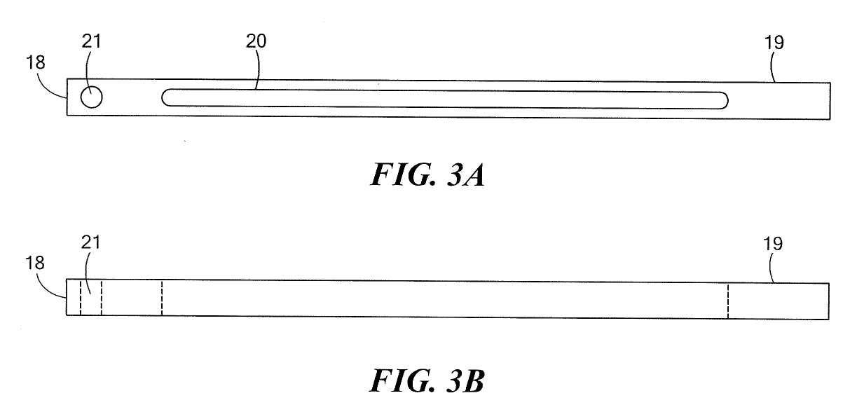

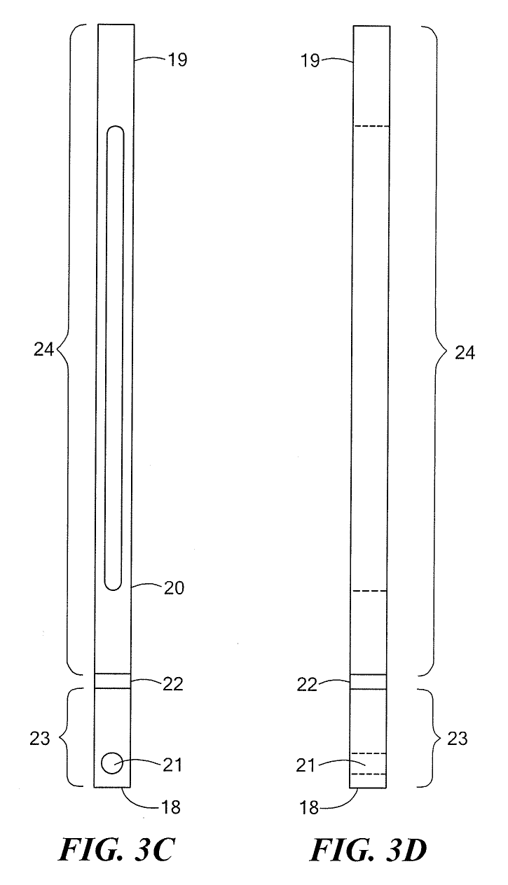

The slider rod 3 has a first end 18 and second end 19 and a longitudinal slot 20 (see FIG. 3A for slot 20) running part of the length of the rod, and slides horizontally within the hollow elongated body 4. There is also a hole 21 (see FIG. 3B) in first end 18 wherein the movable-stylus guide pin 10 slides vertically within the cannulated movable stylus 1, and is attached transversely through the first end 18 of the slider rod 3. Encircling the slider rod 3 is a pointer 5 that slides with the slider rod 3 but whose position along the slider rod 3 may also be adjusted manually. The slider rod may also be threaded on the first end to receive the movable stylus 1.

As shown in FIGS. 1B, 3C, and 3D, some embodiments of the slider rod 3 may include a rotatable coupling 22 that allows one end of the slider rod 3 to rotate relative to the opposing end. The rotatable coupling 22 may be located between the end of the longitudinal slot 20 at the first end 18 of the slider rod 3 and the first end of the slider rod 18. In this orientation, the rotatable coupling allows portion 23 of the slider rod 3 to rotate relative to portion 24 of the slider rod 3.

The plane of the rotatable coupling 22 may be perpendicular to the axis of the slider rod 3. This orientation segregates side 18 from side 19. The segregation of side 18 and 19 functionally permits rotation of the slider rod portions 23 and 24 independently from one another in the plane of the rotatable coupling 22.

Although the slider rod 3 with the rotatable coupling 22 is described above as a one piece structure, the slider rod 3 and rotatable coupling 22 structure can be any number of elements. For example, the slider rod ends 18 and 19 can be separate and distinct elements. Additionally, the rotatable coupling 22 can also be a separate element, creating a 3-piece slider rod. In such an embodiment, the rotatable coupling 22 may be a cylinder of flexible material that slides over opposing ends of the two sides 18 and 19. The flexible material allows the two ends 18 and 19 to rotate with respect to each other. In other embodiments, the rotatable coupling 22 can be a more complex, multi-component element, such as a bushing or bearing, creating a slider rod 3 and rotatable coupling 22 structure with greater than three elements.

The rotatable coupling 22 can be any number of elements that permit relative motion between portions 22 and 23 of the slider rod 3. For example, the rotatable coupling 22 may be a bushing, thrust bearing, rotary bearing, or other assembly that allows relative rotational motion between components.

The cannulated fixed stylus 2 is attached transversely through the first end 16 of the isometer body 4 and has a corresponding fixed-stylus guide pin 8 that slides vertically within the cannulated fixed stylus 2, through the first end 16 of the isometer body 4. A fixed-stylus set screw 7 is attached near the open-end top of the fixed stylus 2 for immobilizing and releasing the fixed-stylus guide pin 8 within the fixed stylus 2.

The cannulated movable stylus 1 is attached transversely through the first end 18 of the slider rod 3 and has a corresponding movable-stylus guide pin 10 that slides vertically within the cannulated movable stylus 1, through the first end 18 of the slider rod 3. A movable-stylus set screw 9 is attached near the open-end top of the movable stylus 1 for immobilizing and releasing the movable-stylus guide pin 10 within the movable stylus 2.

There may also be a cannulated handle 6 attached to the isometer body 4, aligned in such a way that the fixed-stylus guide pin 8 can be directed through the handle cannulation, though the isometer body 4, and through the slider rod 3, and through the fixed stylus 2.

The cannulated elongated body may also have a side window opening 12 for viewing movement of the pointer 5 along the slider rod 3.

During intra-articular ACL or PCL reconstruction, first a lateral incision is made through the subcutaneous tissue from the lateral femoral epicondyle to Gerdy's Tubercle. The fascia lata is divided longitudinally. Gerdy's tubercle is identified along with the site just posterior to the insertion of the lateral collateral ligament on the femur as the sites of the reconstruction tunnels as shown in FIG. 1A. The isometer is then used.

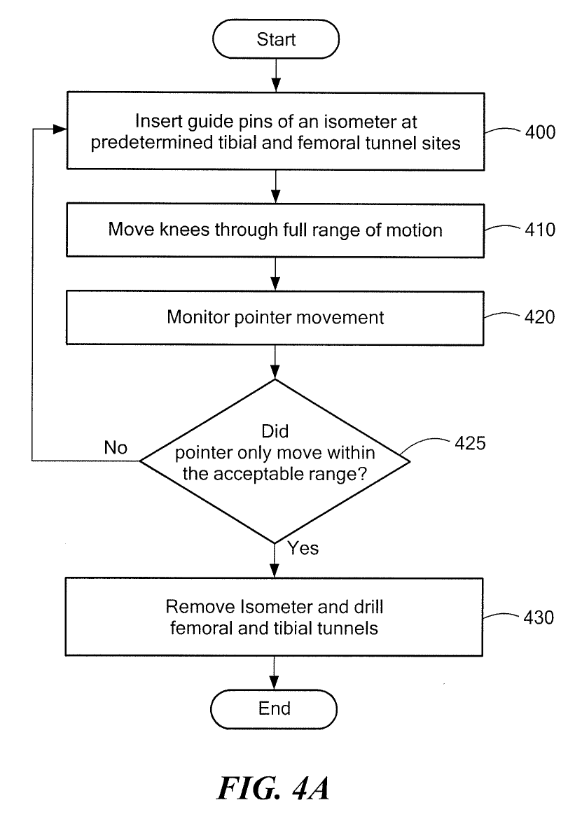

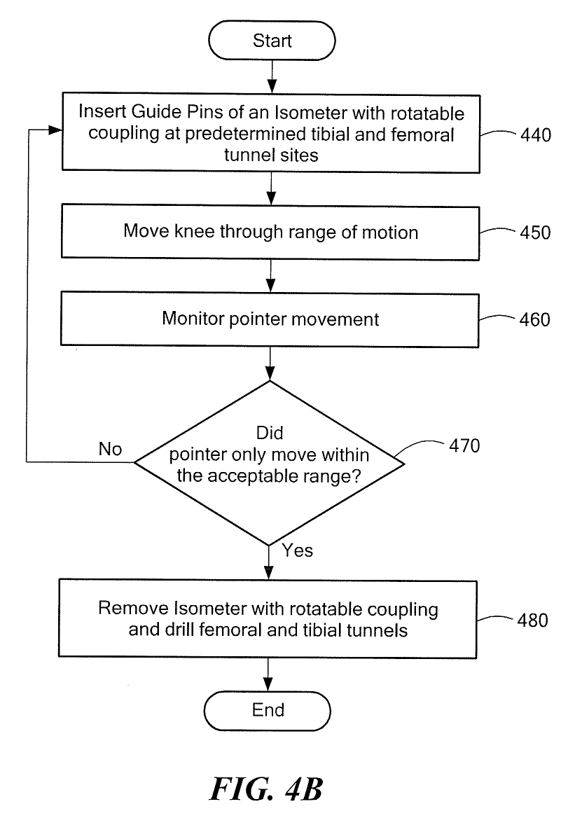

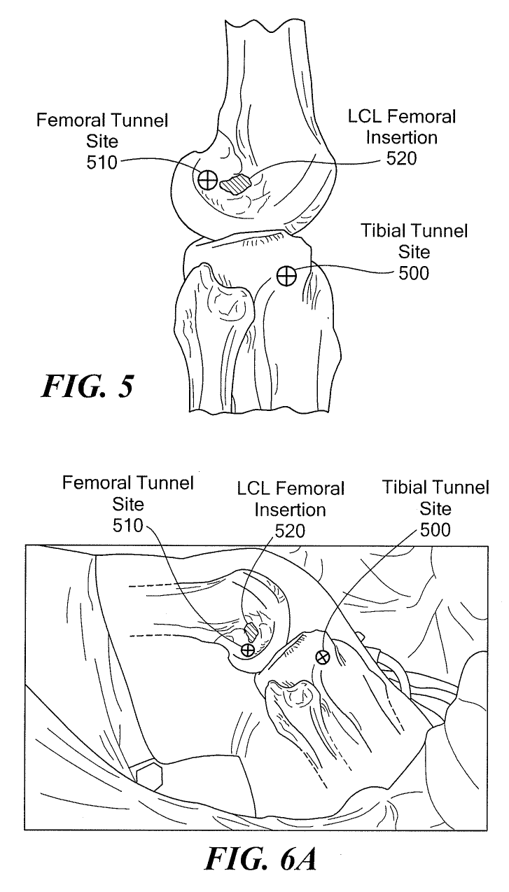

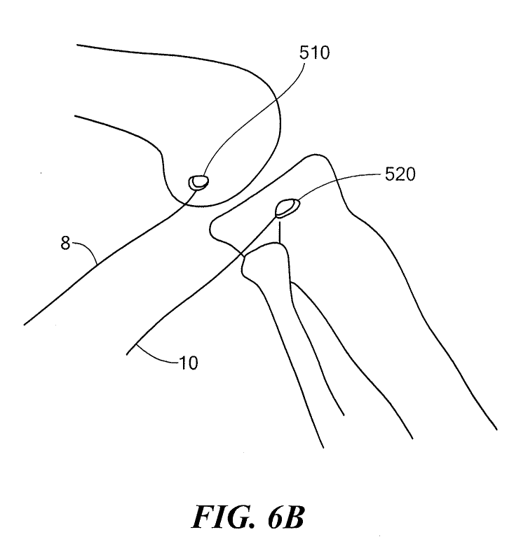

FIG. 4A provides a flow chart of the method of using the isometer to determine a position of isometry for restoring lateral stability during ACL or PCL reconstruction. FIG. 4B provides a flow chart of the method of using the isometer with a rotatable member to determine a position of isometry. As can be seen in FIGS. 4A and 4B, the methods are substantially the same. First, the fixed- and movable-stylus guide pins 8 and 10 are initially directed into the corresponding fixed and movable styli 2 and 1 and advanced until the guide pins protrude beyond the corresponding stylus tips preferably ¼ inch. The guide pins 8 and 10 are then immobilized within the corresponding styli 2 and 1 by tightening the corresponding set screws 7 and 9. The immobilized guide pins 8 and 10 are then positioned over previously identified lateral reconstruction tunnel sites through skin incisions such that the fixed stylus 2 and its guide pin 8 are positioned over a femoral tunnel site and the movable stylus 1 and its guide pin 10 are positioned over a tibial tunnel site (Step 400/440). The initial tunnel placement sites are identified as Gerdy's tubercle on the tibia 500 along with the site 510 just posterior to the insertion of the lateral collateral ligament 520 on the femur as shown in FIG. 5 and FIG. 6A.

The placement sites of the stylus and guide pins may be reversed, such that the fixed stylus 2 and its guide pin 8 are positioned over previously identified tibial tunnel sites and the movable stylus 1 and its guide pin 10 are positioned over previously identified femoral tunnel sites.

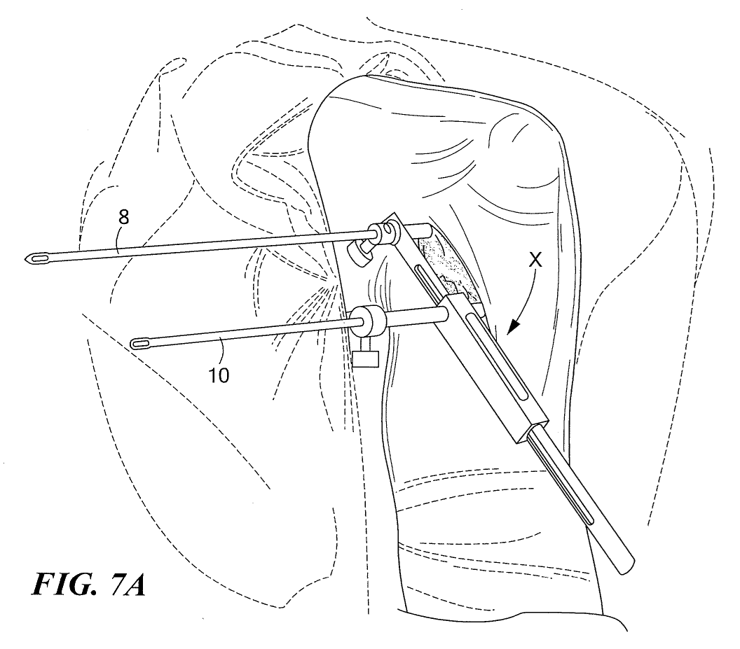

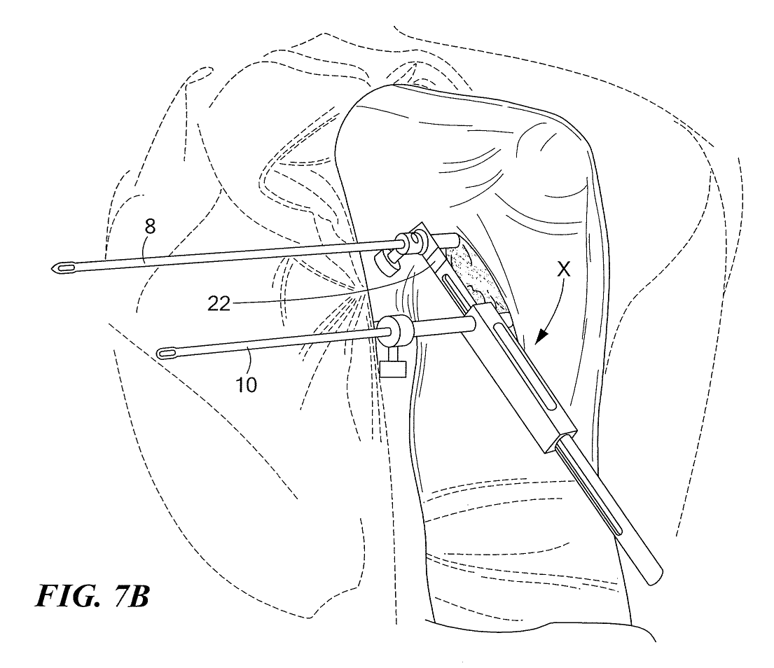

The guide pins 8 and 10 are then driven into the bone tunnel sites, preferably using a light mallet. The pins are driven preferably to a depth of about ⅛ to ¼ inch. In another embodiment, the pins are first driven into the femoral tunnel site 510 and tibial tunnel site 500 and then the isometer is placed on the pins (see FIG. 6B). Although difficult to achieve, it is important that the axes of the guide pins 8 and 10 remain as close to parallel as possible. In FIGS. 7A and 7B, the guide pins 8 and 10 are substantially parallel. As deviation from parallel increases, the risk of isometer impingement increases. The risk of impingement is reduced in embodiments containing a rotatable coupling 22 on the slider rod 3. As discussed above, the rotatable coupling 22 allows portions 23 and 24 of the slider rod 3 to rotate freely relative to one another. The rotatable coupling 22 allows 360 degrees of rotation such that portion 23 is free to rotate while portion 24 of the slider rod is secured. The rotatable coupling 22 obviates impingement created by a rigid slider rod 3 when the guide pins 8 and 10 are not perfectly parallel. Therefore, the rotatable coupling 22 facilitates accurate isometer testing throughout the entire range of motion (ROM) cycling even if the guide pins axes are not perfectly parallel.

FIG. 7A shows the guide pins 8 and 10 in place during surgery with the isometer X positioned over the guide pins. Similarly, FIG. 7B shows the guide pins 8 and 10 in place during surgery with isometer X having the rotatable coupling 22. The isometer position is maintained as the knee is then flexed and extended slowly throughout its full range of motion in the anterior-posterior axis in the sagittal plane. (Step 410/450) This test of the range of motion is used to find a substantially isometric point. If the points are not substantially isometric, the graft will be too loose in one area of knee flexion or too tight in another area of flexion. If the graft is too tight the knee will either not be able to move fully or the graft will stretch. During this test of the full movement of knee motion, the movement of pointer 5 is monitored in relation to the isometer body 4 (Step 420/460). The pointer 5 movement may be measured during the changes in the range of motion (ROM). Typically, the largest changes occur between the middle of the range of motion and one extreme of the range of motion (e.g., full extension or full flexion). The amount of movement is measured as an absolute value. In other words, the amount of movement is measured as the difference between the maximum and minimum distances between the guide pins 8 and 10 during the range of motion test. If the guide pins 8 and 10 were placed perfectly, there would be no change in the pointer location throughout the ROM.

The isometer body may be provided with markings which are indicative of distance. The surgeon then assess whether the movement of the pointer is acceptable (Step 425/470). A pointer movement maximum of less than 2 mm is preferred and a value of between 2 mm and 3 mm is an indication that the positions of the pin are approximately at isometric locations. As a result, the markings on the isometer body are preferably in increments of 1 mm or less. Further, markings which indicate the boundary of acceptable range of movement may be provided in place of or in addition to the markings which are indicative of distance. If the movement of the pointer is acceptable, the isometer is removed as explained below, leaving the guide pins. A cannulated drill is then seated on the guide pins and the tibial and femoral tunnels are drilled (Step 430/480). If the movement of pointer 5 is not acceptable, for example more than 3 mm, the movable-stylus guide pin 10 and/or fixed-stylus guide pin 8 is released by loosening corresponding set screw(s) 9 and/or 7, and then re-positioned into a different tibial or femoral tunnel sites. The knee is again put through a full range of motion and the movement of pointer 5 again monitored in relation to the isometer body 4. This re-positioning procedure for the movable stylus 2 and/or the fixed stylus 1 and the corresponding guide pins 10 and 8 may be repeated several time altering the site methodically around the originally chosen site and if after having repeated this process numerous times with no success, only then is the tibial site at Gerdy's tubercle considered for adjustment and the femoral sites are retested against a secondly selected Gerdy's tubercle site.

Once pointer movement 5 is acceptably small indicating that isometry has been determined, the set screws 7 and 9 are loosened to release the guide pins 8 and 10 from their corresponding styli 1 and 2, and the isometer is then removed, leaving the guide pins 8 and 10 in place. The femoral pin is drilled through the femoral cortex exiting medially and sufficiently proximal to avoid the femoral tunnel of the previously reconstructed ACL. The tibial pin is advanced distally and medially in order to avoid the tibial tunnel (if present in an accompanying ACL reconstruction) until it exits the skin.

The guide pins are then used to direct placement of the drill, but once the tunnels are created the guide pins are removed and the reconstructive surgery is then completed.

In a different variation of the methodology of using the isometer, guide pins are first inserted into the femur and tibia at the proposed positions of the femoral and tibial tunnel sites. The styli of the isometer are then aligned with and placed over the guide pins and the guide pins are locked in position using a locking mechanism so that the guide pins are firmly held in position during movement of the knee. The knee is then moved through a normal flexion range of motion as described above and the subsequent steps of the methodology are the same.

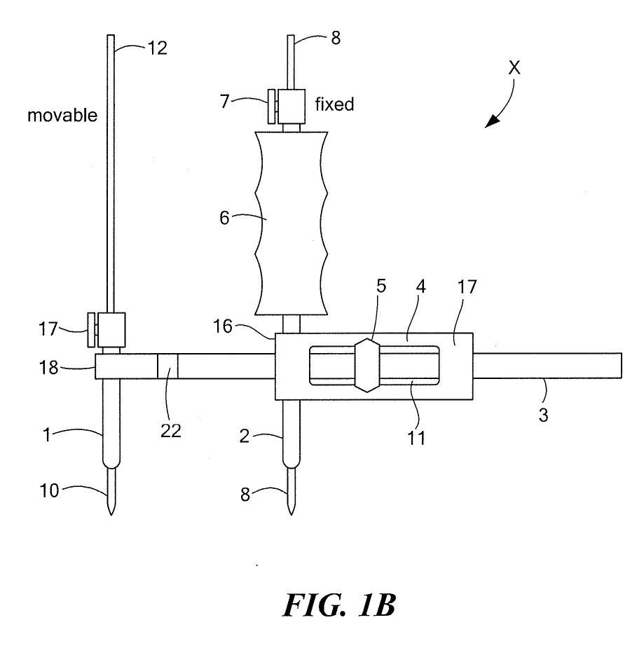

Referring now to FIG. 2 and elements of FIG. 1, an enlarged view of elongated cannulated body 4, can be seen, with openings 13 for encasing slider rod 3 of FIG. 1 and opening 14 in the first end of body 4 for the cannulated fixed stylus 2 that is attached transversely through the first end of the isometer body 4 and further has a corresponding fixed-stylus guide pin 8 that slides vertically within the cannulated fixed stylus 2, and thus through the first end of the isometer body 4. The cannulated elongated body may also have a side window opening 12 for viewing movement of the pointer 5 along the slider rod 3.

FIG. 3A shows a top view and FIG. 3B shows side view of slider rod 3, and typical dimensions that may be used in accordance with the present invention. The slider rod 3 includes slot 20 so that the slider rod 3 slides around fixed stylus 2 within cannulated body 4. The hole 21 of the slider rod 3 allows the movable stylus 1 to be inserted and held in position relative to the slider rod 3 during flexing of the knee.

It should be understood by one of ordinary skill in the art that each stylus, may include a sharp point for insertion into a bone and that the isometer could be used without guide pins. In such an embodiment, the markings made in the bone by the stylus would be used for either subsequent insertion of a guide pin and used in conjunction with a cannulated drill or the markings could be used with a manual or power drill. It should also be understood that the use of guide pins is preferred since guide pins provide additional precision in the positioning of the femoral and tibial tunnels, since the cannulated drill drills at precisely the position of the guide pin whereas a manual drill may walk away from the marking and therefore provide a less precise tunnel location.

The present invention provides both a surgical device and a reproducible and standardized method for determining lateral rotary isometry in combined ALR/ACL reconstruction. The isometer of the present invention also essentially eliminates the technique-intensive and time-consuming problems inherent in “free-hand” determinations of lateral rotary isometry.

Although various exemplary embodiments of the invention have been disclosed, it should be apparent to those skilled in the art that various changes and modifications can be made that will achieve some of the advantages of the invention without departing from the true scope of the invention. These and other obvious modifications are intended to be covered by the appended claims.

What is claimed is:

1. An isometer for use during lateral augmentation or reconstruction surgery for determining if a first tunnel site and a second tunnel site are isometric, the isometer comprising:
a cannulated body;
a slider rod having a first end and a second end and wherein the slider rod passes through the cannulated body and is free to slide within the cannulated body;
a first stylus attached to the cannulated body; and
a second stylus attached to the slider.

2. The isometer according to claim 1, wherein the slider rod includes a rotatable coupling located between the first end and second end, wherein the rotatable coupling allows the first end and second end to rotate relative to each other about an axis.

3. The isometer according to claim 2, wherein the axis of rotation lies along a length of the slider rod.

4. The isometer according to claim 2, wherein the rotatable coupling allows the first end and second end to rotate less than 360 degrees.

5. The isometer according to claim 2, wherein the rotatable coupling allows the first end and the second end to rotate 360 degrees.

5. The isometer according to claim 2, wherein the rotatable coupling is a bushing.

6. The isometer according to claim 1 wherein the body has an opening that exposes the slider rod.

7. The isometer according to claim 6, wherein the slider rod includes a pointer which is visible through the opening on the side of the body.

8. The isometer according to claim 7, wherein the body has indicia for measuring position of the pointer attached to the slider.

9. The isometer according to claim I wherein the first stylus and second stylus include points for insertion into a bone.

10. The isometer according to claim 1, wherein the first stylus and second stylus are cannulated.

11. The isometer according to claim 10 further comprising:
a first guide pin passing through the first cannulated stylus; and
a second guide pin passing through the second cannulated stylus.

12. The isometer according to claim 11 wherein the first guide pin and the second guide pin include a first point and a second point, respectively, for inserting into a bone.

13. The isometer according to claim 11, wherein the first stylus and second stylus each include a restraint for securing the first and second guide pins in the first and second cannulated stylus.

14. The isometer according to claim 13, wherein the restraints are configured to release the first and second styli when the guide pins are inserted into a bone such that the isometer can be removed, leaving the guide pins inserted in the bone.

15. The isometer according to claim 14, wherein the restraints include a set screw which can be tightened to secure the first and second guide pins and loosened to release the first and second guide pins.

16. The isometer according to claim 1, wherein the first stylus passes through a hole at the first end of the sliding rod and the second stylus passes through a slot running part of the length of the slider rod such that the slider rod can slide around the second stylus.

17. The isometer according to claim 16 wherein the slider rod includes a rotatable coupling located between the hole at the first end and an end of the slot.

18. A method of using an isometer to determine a position of isometry during knee surgery comprising:
a) inserting a first guide pin into a moveable stylus such that the tip of the guide pin protrudes past an end of the moveable stylus;
b) inserting a second guide pin into a stationary stylus such that the tip of the guide pin protrudes past an end of the stationary stylus;
c) securing the first and second guide pins within the moveable and stationary styli;
d) positioning the isometer such that the guide pins are located over previously identified tunnel sites;
e) driving the guide pins into the tunnel sites;
f) flexing and extending the knee such that a slider rod of the isometer moves relative to an isometer body and wherein the slider rod includes a rotatable coupling that permits a first end and a second end of the slider arm to rotate relative to one another, compensating for any deviation from parallel between the first and second guide pins' axes; and
g) monitoring the movement of a pointer located on a slider rod of the isometer in relation to the isometer body.

19. The method of using an isometer according to claim 18, wherein the first guide pin is positioned over a tunnel site located in the femur and the second guide pin is located over a tunnel site located in the tibia.

20. The method of using an isometer according to claim 18, wherein the first guide pin is positioned over a tunnel site located in the tibia and the second guide pin is located over a tunnel site located in the tibia.

21. The method of using an isometer according to claim 18, wherein the guide pins are driven into the tunnel sites to a depth of ⅛ to ¼ of an inch.

22. The method of using an isometer according to claim 18 further comprising:
re-positioning at least one of the first and second styli if the movement is unacceptably large; and
repeating steps d) through g) until the pointer movement is acceptable.

23. The method of using an isometer according to claim 18 further comprising:
if the pointer movement is acceptably small, unsecuring the guide pins in the moveable and stationary styli; and
removing the isometer, leaving the guide pins in place.

24. The method of using an isometer according to claim 23 wherein the pointer movement is acceptably small when the pointer movement is 3 mm or less.

25. A method of using an isometer to determine a position of isometry during knee surgery comprising:
a) driving a first guide pin into a predetermined tunnel site in a femur;
b) driving a second guide pin into a predetermined tunnel site in a tibia
c) placing the isometer over the guide pins such that a moveable stylus located on the isometer slides over one guide pin and a stationary stylus located on the isometer slides over the other guide pin such that the tip of the guide pins protrude past the ends of the styli;
d) securing the first and second guide pins within the moveable and stationary styli;
e) flexing and extending the knee such that a slider rod of the isometer moves relative to an isometer body and wherein the slider rod includes a rotatable coupling that permits a first end and a second end of the slider arm to rotate relative to one another, compensating for any deviation from parallel between the first and second guide pin; and
f) monitoring the movement of a pointer located on a slider arm of the isometer in relation to the isometer body.

26. The method of using an isometer according to claim 25, wherein the moveable stylus is placed over the first guide pin and the stationary stylus is placed over the second guide pin.

27. The method of using an isometer according to claim 28, wherein the stationary stylus is places over the first guide pin and the moveable stylus is placed over the second guide pin.

28. The method of using an isometer according to claim 25, wherein the guide pins are driven into the tunnel sites to a depth of ⅛ to ¼ of an inch.

29. The method of using an isometer according to claim 25 further comprising:
re-positioning at least one of the first and second guide pins if the movement is unacceptably large; and
repeating steps a) through e) until the pointer movement is acceptable.

30. The method of using an isometer according to claim 25 further comprising:
if the pointer movement is acceptably small, unsecuring the guide pins in the moveable and stationary styli; and
removing the isometer, leaving the guide pins in place.

31. The method of using an isometer according to claim 30 wherein the pointer movement is acceptably small when the pointer movement is 3 mm or less.
